(19) United States
(12) Patent Application Publication
(10) Pub. No.: US 2025/0369031 A1
(43) Pub. Date: Dec. 4, 2025

(54) METHODS OF DETERMINING CERULOPLASMIN ACTIVITY AND APPLICATIONS THEREOF

(71) Applicant: ACADEMIA SINICA, Taipei City (TW)

(72) Inventor: Wei-Chieh CHENG, Taipei City (TW)

(73) Assignee: ACADEMIA SINICA, Taipei City (TW)

(21) Appl. No.: 18/876,892

(22) PCT Filed: Jun. 20, 2023
(86) PCT No.: PCT/US2023/025725

§ 371 Date: Dec. 19, 2024

Related U.S. Application Data
(60) Provisional application No. 63/353,742, filed on Jun. 20, 2022.

(51) Int. Cl. C12Q 1/26

(52) U.S. Cl. CPC C12Q 1/26; C12Y 116/03001; G01N 2333/90287; G01N 2800/2814

(57) ABSTRACT

Disclosed is a method of determining ceruloplasmin activity using a mass spectrometry technique. The method comprises reacting a biological sample containing ceruloplasmin with a compound of formula I to produce an oxidized product of the compound, measuring a level of the oxidized product by a mass spectrometry technique, and determining a level of ceruloplasmin activity based on the level (amount) of the oxidized product.

### RELATED APPLICATIONS

This application claims the benefit of U.S. provisional application No. 63/353,742, filed Jun. 20, 2022 under 35 U.S.C. § 119, the entire content of which is incorporated herein by reference.

### TECHNOLOGY FIELD

The present invention relates to a method of determining ceruloplasmin activity and applications thereof. In particular, the present invention provides a compound of formula I for use as a substrate of ceruloplasmin which is applicable in a method of determining ceruloplasmin activity and/or predicting the risk of a disease or condition related to ceruloplasmin activity in a subject by using a mass spectrometry technique. Specifically, the method of the present invention requires trace amounts of a biological sample to be tested and achieves desired reliability and sensitivity.

### BACKGROUND OF THE INVENTION

Human serum contains many types of oxidase, including glutathione peroxidase, myeloperoxidase (MPO), ferroxidase, cholesterol oxidase, monoamine oxidase, etc. These oxidases play important roles in metabolism. For examples, glutathione plays important roles in antioxidant defense, nutrient metabolism, and regulation of cellular events (including gene expression, DNA and protein synthesis, cell proliferation and apoptosis, signal transduction, cytokine production and immune response, and protein glutathionylation).¹ MPO is one of the key components of neutrophil extracellular traps (NETs) formed during neutrophil-specific cell death.² Ferroxidase is important for iron metabolism, which catalyzed the oxidation of Fe²⁺ to Fe³⁺. This is a key reaction as it permits iron to be bussed to various organs.^{3,4} Among all oxidases, ceruloplasmin (Cp), a multicopper ferroxidase involved in the oxidation of selected substrates, is abundant in the serum.⁵

Cp is an essential ferroxidase that plays an important part in copper and iron metabolism. It contains six copper atoms bound to specific copper-binding sites, which function as the critical catalysts. It oxidizes Fe²⁺ into Fe³⁺ by coupling with two-electron reduction of oxygen to water and involving electron transfer from the type I copper sites (where the iron binds) to the trinuclear copper cluster (where the oxygen binds).^{6,7} The oxidation of ferrous iron by ceruloplasmin allows the oxidized iron to bind with iron chaperone transferrin for entry into the systemic iron cycle and delivery among tissues. The mutations in those proteins required to shuttle copper for holoceruloplasmin (copper-dependent ferroxidase) biosynthesis cause the accumulation of apoceruloplasmin, which is lacking copper and ferroxidase activity.⁸⁻¹⁰ Several diseases may occur due to the deficit of oxidase activity, such as Wilson's disease^{5,6}, Alzheimer's disease³, and Parkinson's disease.¹¹

At present the colorimetric enzymatic assay is commonly used in the measurement of serum oxidase activity. Aromatic diamines, such as p-phenylenediamine, are usually utilized in the colorimetric assay since they can be oxidized by serum oxidases.¹² An assay using the enzymatic oxidation of o-dianisidine dihydrochloride developed by Schosinsky et al. resulted in formation of a photogenic oxidized product that can easily be measured by UV/Vis photometric readers.^{13,14} However, this method requires a serum sample to be tested in a certain amount and the oxidized product is a mixture of unclear polymeric forms¹⁵, indicating that the sensitivity of a colorimetric assay is constrained and its stability is also a challenge.

Liquid chromatography-tandem mass spectrometry (LC-MS/MS) offers a highly sensitive detection technique. This technique of mass spectrometry to study enzyme activity with a proper enzyme substrate to quantitatively detect a product has been reported.¹³⁻¹⁵ However, the analysis of serum oxidases activity by using LC-MS/MS based assay remains to be developed since no reliable substrates for measuring serum Cp activity are available currently.¹⁶

### SUMMARY OF THE INVENTION

The present invention is at least based on the finding that a compound of formula I is useful as a substrate of ceruloplasmin (Cp) for measuring serum Cp activity by a mass spectrometry technology. Therefore, the present invention provides a spectrometry technology-based assay for determining serum Cp activity using a compound of formula I as the substrate. The assay is applicable in clinical applications for predicting the risk of a disease related to ceruloplasmin activity in a subject, especially in a child and an infant. The assay of the present invention requires trace amounts of a biological sample to be tested and achieves desired reliability and sensitivity.

In particular, in one aspect, the present invention provides a method of determining ceruloplasmin activity, comprising:
(i) contacting a biological sample containing ceruloplasmin with a compound of formula I under a condition such that the compound reacts with the ceruloplasmin to produce an oxidized product of the compound

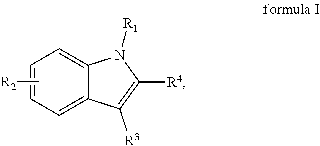

wherein
R₁ is hydrogen, an alkyl group, an alcohol group, an alkoxy group or an alkyl ether group containing one or more oxyethylene units;
R₂ is hydrogen or hydroxyl; and
R₃ and R₄, the same or different, are independently hydrogen or an alkylamine group, or
R₃ and R₄ together form a benzene ring fused to the nitrogen-containing bicyclic heteroring, wherein the benzene ring is substituted by —NH₂;
(ii) measuring a level of the oxidized product by a mass spectrometry technique; and
(iii) determining a level of ceruloplasmin activity based on the level of the oxidized product, wherein a decreased level of the oxidized product is indicative of a decreased level of ceruloplasmin activity, and/or an increased level of the oxidized product is indicative of an increased level of ceruloplasmin activity.

In some embodiments, R₁ is hydrogen, an alkyl group, an alcohol group, an alkoxy group or an alkyl ether group containing one or more oxyethylene units; R₂ is hydrogen or hydroxyl; and R₃ and R₄ are independently hydrogen or an alkylamine group.

In some embodiments, R₁ is hydrogen, R₂ is hydroxyl, R₃ is an alkylamine group and R₄ is hydrogen.

In one particular embodiment, the compound is represented by formula Ia

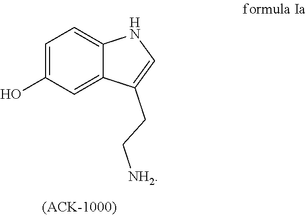 [SMILES: NCCc1c[nH]c2ccc(O)cc12]

In some embodiments, R₁ is hydrogen, an alkyl group, an alcohol group, an alkoxy group or an alkyl ether group containing one or more oxyethylene units; R₂ is hydrogen or hydroxyl; and R₃ and R₄ together form a benzene ring fused to the nitrogen-containing bicyclic heteroring, wherein the benzene ring is substituted by —NH₂.

In some embodiments, R is an alkyl group, an alcohol group or an alkyl ether group containing one or more oxyethylene units; R₂ is hydrogen; and R₃ and R₄ together form a benzene ring fused to the nitrogen-containing bicyclic heteroring, wherein the benzene ring is substituted by —NH₂.

In some particular embodiments, the compound is represented by formula

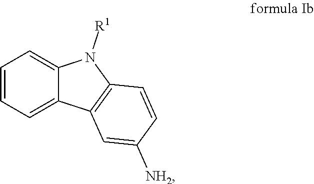

wherein R₁ is —CH₃, —CH₂CH₃, —CH₂CH₂CH₂CH₃, —CH₂CH₂OH, —CH₂CH₂CH₂OH or —CH₂CH₂(OCH₂CH₂)₄OCH₃.

Examples of the compound of formula (Ib) are selected from the group consisting of

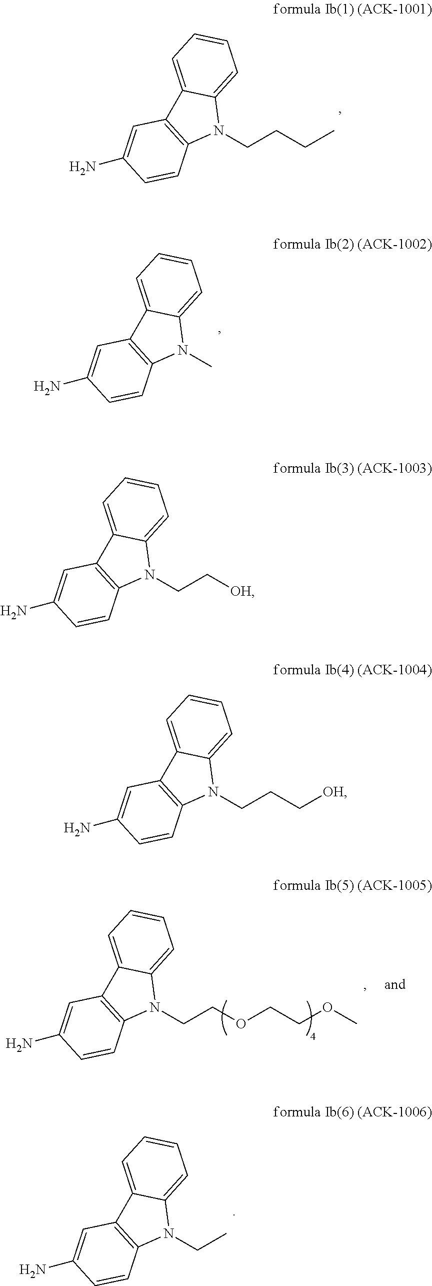

In some embodiments, the spectrometry technique comprises a tandem mass spectrometry (MS/MS) technique. In one example, the spectrometry technique comprises an LC-MS/MS technique.

In some embodiments, the biological sample is a liquid biological sample such as a serum sample or a urine sample.

In some embodiments, the biological sample has a volume of 0.01 μL to 10 μL, for example, 0.05 μL to 5 μL, such as 0.05 μL to 2 μL.

In another aspect, the present invention provides a method for determining ceruloplasmin activity and predicting the risk of a disease related to ceruloplasmin activity in a subject, comprising:
(i) providing a biological sample which is obtained from the subject;
(ii) contacting the biological sample with a compound of formula I as described herein under a condition such that the compound is oxidized to produce an oxidized product of the compound;
(iii) measuring a level of the oxidized product by a mass spectrometry technique; and
(iv) determining a level of ceruloplasmin activity and the likelihood of having a disease or condition related to ceruloplasmin activity of said subject based on the level of the oxidized product, wherein
a decreased level of the oxidized product below a reference level is indicative of deficiency of ceruloplasmin activity and an increased likelihood of having a disease or condition related to deficiency of ceruloplasmin activity; and
an increased level of the oxidized product above a reference level is indicative of excess of ceruloplasmin activity and an increased likelihood of having a disease or condition related to excess of ceruloplasmin activity.

In a further aspect, the present invention provides a kit for performing a method as described herein which comprises a compound of formula I, a reaction buffer, a quenching agent and instructions for using the kit to determine ceruloplasmin activity and/or predicting the risk of a disease related to ceruloplasmin activity in a subject by using a mass spectrometry technique.

The present invention also discloses a compound of formula I for use as a substrate of ceruloplasmin for manufacture of a kit for performing a method of determining ceruloplasmin activity and/or predicting the risk of a disease related to ceruloplasmin activity in a subject by using a mass spectrometry technique. Also disclosed is use of a compound of formula I as a substrate of ceruloplasmin for manufacture of a kit for performing a method of determining ceruloplasmin activity and/or predicting the risk of a disease related to ceruloplasmin activity in a subject by using a mass spectrometry technique.

In some embodiments, a disease or condition related to deficiency of ceruloplasmin activity includes aceruloplasminemia, Wilson's disease, Menke's disease, Alzheimer's disease and Parkinson's disease (PD).

In some embodiments, a disease or condition related to excess of ceruloplasmin activity includes acute inflammation, rheumatoid arthritis or infection.

In some embodiments, the subject is a fetus, a neonate, a child, an adolescent or an adult.

The present invention further provides a compound of formula II

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows general concept for screening of in-house chemicals to discover new serum oxidase substrates for Mass analysis.

FIGS. 2A and 2B show the candidate substrates of the present invention and synthesis thereof. FIG. 2A shows the structures of compounds ACK-1000 and ACK-1001. FIG. 2B illustrates the synthesis of a series of modified compound for ACK-1001.compounds as a serum oxidase substrate.

FIG. 3A and FIG. 3B show the results of LC-MS/MS detection of serum oxidases with modified heterocyclic compounds of the present invention, including FIG. 3A for ACK-1001, ACK-1002, ACK-1003, ACK-1004 and ACK-1005, and FIG. 3B for ACK-1006.

FIG. 4 shows the results of the ceruloplasmin (Cp) spike-in analysis. The analysis was examined by the addition of different amount of Cp (20 U/mL) in serum sample (5 μL), incubating with ACK-1006 and the DMSO quenched mixture were detected by LCMS with 100-fold dilution.

FIG. 5 shows the results of the Cp-depletion test. The test was performed by the detection the oxidized product of ACK-1006. Substrate ACK-1006 were incubated with Cp-depleted serum (5 μL) for 30 min at 37° C. Normal adult serum and plasma were controls. After incubation, the DMSO quenched mixture were 100-folds diluted and detected by LCMS.

FIGS. 6A and 6B show the LC-MS/MS based serum oxidase activity assay of the present invention. FIG. 6A shows a flowchart illustrating the steps of the LC-MS/MS based serum oxidase activity assay of the present invention. FIG. 6B shows the results of the LC-MS/MS based serum oxidase activity assay using ACK-1006 as the substrate according to the present invention.

FIG. 7 shows the results of the time-dependent study of the oxidized product formation with ACK-1006 as a serum oxidase substrate.

FIGS. 8A and 8B show the limitations of detection by current methods known in the art. FIG. 8A shows the correlation of serum volume with absorbance obtained by an o-dianisidine dihydrochloride method. FIG. 8B shows the correlation of serum volume with absorbance obtained by a p-phenylenediamine method. The specific activity units of Cp are not reliable in lower volume of serum.

FIGS. 9A to 9D show the limitation of detection of commercially available kits. FIG. 9A and FIG. 9C show the correlation of serum volume with absorbance using the BioVision kit and the Invitrogen kit, respectively. FIG. 9B and FIG. 9D show the Cp activity unit determined by the BioVision kit and the Invitrogen kit, respectively. The results show that the current commercial kits are not reliable in low volume of serum.

FIGS. 10A and 10B show the limitation of detection of the LC-MS/MS based serum oxidase activity assay of the present invention. FIG. 10A show the correlation of serum volume (0.05 μL to 0.5 μL) and MS peak area (oxidative ACK1006) in MS spectrometry according to the present invention. FIG. 10B shows the Cp activity unit determined by the method of the present invention. The results show that the Cp activity unit is stable in extremely lower serum volume conditions (0.05 μL to 0.5 μL).

FIG. 11 shows comparison of Cp activities between Cp-depletion and new born plasma. Substrate ACK-1006 were incubated with new born plasma and Cp-depleted serum (5 μL) for 30 min at 37° C. The DMSO quenched mixture were detected by LCMS without dilution.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

As used herein, the term “comprise” or “comprising” is generally used in the sense of include/including which means permitting the presence of one or more features, ingredients or components. The term “comprise” or “comprising” encompasses the term “consists” or “consisting of.”

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±10% around the cited value.

As used herein, the terms “subject,” “individual” and “patient” refer to any mammalian subject for whom diagnosis, prognosis, treatment, or therapy is desired, particularly humans. Other subjects may include cattle, dogs, cats, guinea pigs, rabbits, rats, mice, horses, and so on.

As used herein, the term “a normal individual” may be used to refer to an individual who is basically in a healthy condition without particular diseases (e.g., Wilson's disease, Alzheimer's disease and Parkinson's disease), and may refer to a single normal/healthy individual or a group of normal/healthy individuals.

As used herein, the term “ceruloplasmin” or the abbreviation “Cp” refers to a ferroxidase enzyme which in humans is encoded by the CP gene. Ceruloplasmin is made in the liver and then secreted into the blood. It contains multiple copper atoms and oxidizes ferrous ion (Fe²⁺) into ferric iron (Fe³⁺) to allow the transport of iron. A blood test can determine ceruloplasmin's amount or activity in the blood. For example, the normal values (reference range) for ceruloplasmin's amount in the blood have been investigated and are in general between 20 and 50 mg/dL which can vary with different populations. Measurement of ceruloplasmin in the blood can be used to evaluate and manage of copper/iron-related diseases/conditions. A low amount or low activity of ceruloplasmin can possibility indicate that the person suffers from, for example, aceruloplasminemia characterized by iron accumulation in the brain and other organs which is caused by lack of ceruloplasmin ferroxidase activity due to mutation of the corresponding CP gene; Wilson's disease in which excessive amounts of copper accumulate in the body; and Menke's disease in which copper absorption is decreased from the intestine, causing systemic copper and ceruloplasmin deficiencies. A low amount or low activity of ceruloplasmin is also correlated with some neurodegenerative diseases such as Alzheimer's disease and Parkinson's disease (PD). On the other hand, increased levels of ceruloplasmin may indicate acute inflammation, rheumatoid arthritis or infection.

According to the present invention, a compound of formula I is used as an enzymatic substrate of ceruloplasmin in a mass spectrometry technique. The compound of formula I is of the structure as follows.

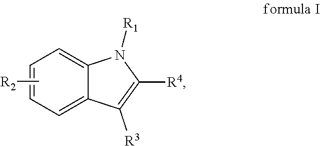

wherein
R₁ is hydrogen, an alkyl group, an alcohol group, an alkoxy group or an alkyl ether group containing one or more oxyethylene units;
R₂ is hydrogen or hydroxyl; and
R₃ and R₄, are independently hydrogen or an alkylamine group, or
R₃ and R₄ together form a benzene ring fused to the nitrogen-containing bicyclic heteroring, wherein the benzene ring is substituted by —NH₂.

As used herein, the term “alkyl” refers to an aliphatic hydrocarbon chain and includes straight and branched chains. An alkyl group may be a C₁-C₁₂ alkyl such as a C₁-C₁₀ alkyl, a C₁-C₈ alkyl, C₁-C₆ alkyl, C₁-C₄ alkyl and C₁-C₃ alkyl. Examples of alkyl, include but are not limited to, methyl, ethyl, n-propyl, isopropyl, n-butyl, n-pentyl, and isobutyl, and the like.

As used herein, an “alcohol group” refers to a hydrocarbon group that includes at least one hydroxy group. An alcohol group may be represented by —ROH wherein R is a C₁-C₁₂ alkylene such as a C₁-C₁₀ alkylene, a C₁-C₈ alkylene, C₁-C₆ alkylene, C₁-C₄ alkylene and C₁-C₃ alkylene. Examples of “alkylene” include, but are not limited to, methylene, ethylene, n-propylene, n-butylene, and the like.

As used herein, the term “alkoxyl” refers to an alkyl group, as defined above, having an oxygen radical attached thereto. Examples of alkoxyl include, but are not limited to, methoxy, ethoxy, n-propoxy, isopropoxy, n-butoxy, iso-butoxy, sec-butoxy, and tert-butoxy.

As used herein, the term “alkyl ether” refers to an alkyl group having at least one oxygen incorporated into the alkyl chain.

As used herein, the term “oxyethylene units” can be represented by —(O—CH₂—CH₂)—ₙ wherein n can be from 1 to 8, e.g., 1 to 7, 1 to 6, 1 to 5, or 1 to 4.

As used herein, the term “an alkyl ether group containing one or more oxyethylene units” may be represented by formula of —(CH₂)ₘ(OCH₂CH₂)ₙ OCH₃, wherein m and n, the same or different, are independently an integer from 1 to 5, e.g., 1, 2, 3, 4 or 5. In some embodiments, m is 2 and n is 4.

As used herein, the term “alkylamine” refers to an alkyl-NH₂ group.

In some embodiments, R₁ is hydrogen, an C₁-C₁₂ alkyl group, an C₁-C₁₂ alcohol group, an C₁-C₁₂ alkoxy group or an alkyl ether group containing one or more oxyethylene units represented by formula of —(CH₂)ₘ(OCH₂CH₂)ₙ OCH₃ wherein m and n, the same or different, are independently an integer from 1 to 5, e.g., 1, 2, 3, 4 or 5; R₂ is hydrogen or hydroxyl; and R₃ and R₄, the same or different, are independently hydrogen or an C₁-C₁₂ alkylamine group.

In some embodiments, R₁ is hydrogen, R₂ is hydroxyl, R₃ is an C₁-C₁₂ alkylamine group and R₄ is hydrogen.

In some particular embodiments, the compound of the present invention is represented by formula Ia

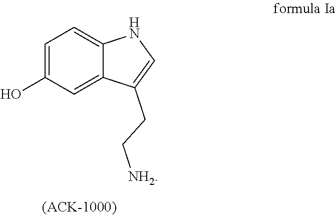 [SMILES: NCCc1c[nH]c2ccc(O)cc12]

In some embodiments, R₁ is hydrogen, an C₁-C₁₂ alkyl group, an C₁-C₁₂ alcohol group, an C₁-C₁₂ alkoxy group or an alkyl ether group containing one or more oxyethylene units represented by formula of —(CH₂)ₘ(OCH₂CH₂)ₙ OCH₃ wherein m and n, the same or different, are independently an integer from 1 to 5, e.g., 1, 2, 3, 4 or 5; R₂ is hydrogen or hydroxyl; and R₃ and R₄ together form a benzene ring fused to the nitrogen-containing bicyclic heteroring, wherein the benzene ring is substituted by —NH₂.

In some embodiments, R₁ is an C₁-C₁₂ alkyl group, an C₁-C₁₂ alcohol group or an alkyl ether group containing one or more oxyethylene units represented by formula of —(CH₂)ₘ(OCH₂CH₂)ₙ OCH₃ wherein m and n, the same or different, are independently an integer from 1 to 5, e.g., 1, 2, 3, 4 or 5; Rz is hydrogen; and R₃ and R₄ together form a benzene ring fused to the nitrogen-containing bicyclic heteroring, wherein the benzene ring is substituted by —NH₂.

In some certain embodiments, R₁ is selected from the group consisting of —CH₃, —CH₂CH₃, —CH₂CH₂CH₂CH₃, —CH₂CH₂OH, —CH₂CH₂CH₂OH and —CH₂CH₂(OCH₂CH₂)₄OCH₃.

In some particular embodiments, the compound of the present invention is represented by formula Ib

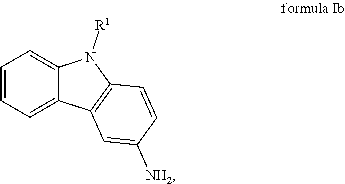

Some examples of the compound of formula I are provided in Table 1 below.

TABLE 1
Examples of commpound of formula I

| Name | Structure |
|---|---|
| ACK-1000 | 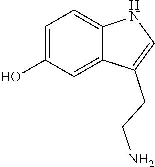 [SMILES: NCCc1c[nH]c2ccc(O)cc12] |
| | formula Ia |
| ACK-1001 | 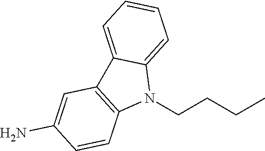 [SMILES: CCCCn1c2ccccc2c2cc(N)ccc21] |
| | formula Ib(1) |
| ACK-1002 | 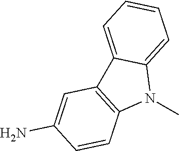 |
| | formula Ib(2) |
| ACK-1003 | 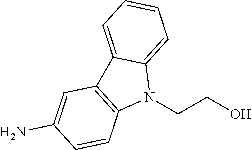 [SMILES: Nc1ccc2c(c1)c1ccccc1n2CCO] |
| | formula Ib(3) |
| ACK-1004 | 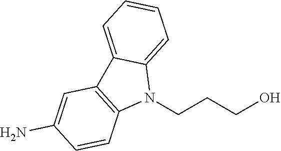 [SMILES: Nc1ccc2c(c1)c1ccccc1n2CCCO] |
| | formula Ib(4) |
| ACK-1005 | 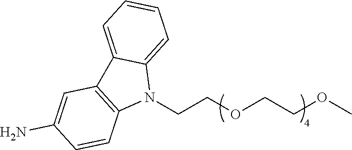 |
| | formula Ib(5) |
| ACK-1006 | 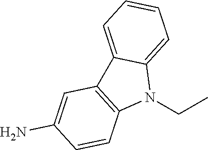 [SMILES: CCn1c2ccccc2c2cc(N)ccc21] |
| | formula Ib(6) |

According to the present invention, the compound of the present invention can be oxidized by ceruloplasmin to an oxidized product. In some embodiments, the oxidized compound reacts with the original (unoxidized) compounds and forms a polymeric (e.g. dimeric or trimeric) structure. In some embodiments, the resultant oxidized product in a polymeric structure is of formula II

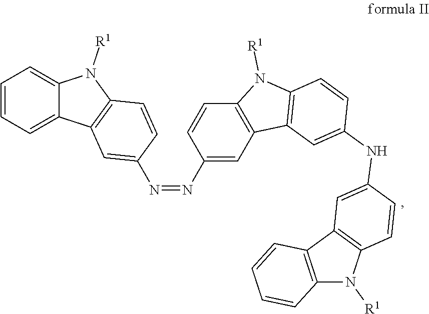

wherein R¹ is as defined as R₁ as described herein. Certain examples of the resultant oxidized product in a polymeric structure are provided as follows.

TABLE 2
Examples of resultant oxidized product

| Name | Structure |
|---|---|
| Oxi- dized ACK- 1001 | Formula II 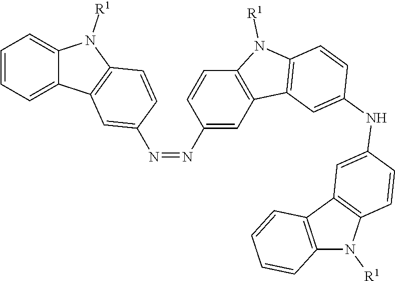 |
| | or isomer(s) thereof, where R¹ |
| | is —CH₂CH₂CH₂CH₃. |
| Oxi- dized ACK- 1002 | Formula II 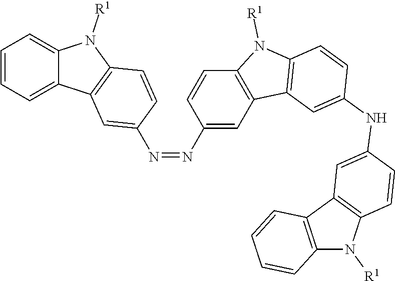 |
| | or isomer(s) thereof, where R¹ |
| | is —CH₃. |
| Oxi- dized ACK- 1003 | Formula II 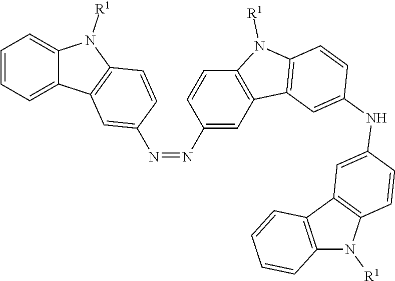 |
| | or isomer(s) thereof, where R¹ is |
| | —CH₂CH₂OH. |
| Oxi- dized ACK- 1004 | Formula II 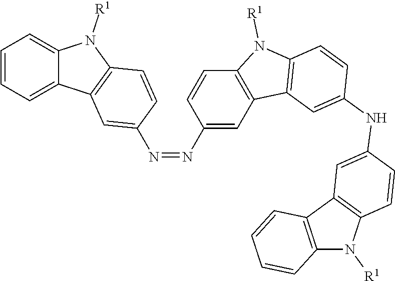 |
| | or isomer(s) thereof, where R¹ |
| | is —CH₂CH₂CH₂OH. |
| Oxi- dized ACK- 1005 | Formula II 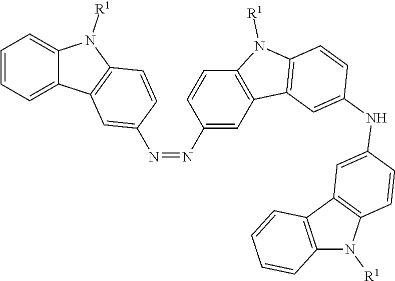 |
| | or isomer(s) thereof, where R¹ is |
| | —CH₂CH₂(OCH₂CH₂)₄OCH₃. |
| Oxi- dized ACK- 1006 | Formula II 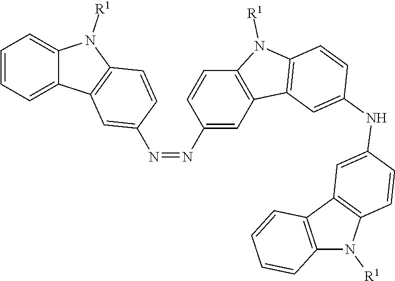 |
| | or isomer(s) thereof, where R¹ |
| | is —CH₂CH₃. |

In step (i) of the method of the invention, the biological sample is reacted with the compound of formula I to obtain a reaction mixture comprising the oxidized product of the compound.

In some embodiments, said compound is allowed to react with the biological sample for about 15 to about 60 minutes at about 35° C. to about 40° C. Preferably, the reaction is allowed for about 20 to about 30 minutes at about 35° C. to about 40° C. More preferably, the reaction is allowed for about 30 minutes at about 35° C. to about 40° C.

In some embodiments, the method of the present invention may further comprise before step (ii) the following step: (i-a) quenching the reaction mixture. Preferably, the reaction mixture is quenched by adding a quenching agent. A particular example of the quenching agent is dimethyl sulfoxide (DMSO).

In some embodiments, the method of the present invention may further comprise before step (ii) the following step: (i-b) collecting from the reaction mixture a solution comprising the oxidized product of the compound. In certain embodiments, the solution is collected by a method comprising: subjecting the quenched reaction mixture to centrifugation, and then collecting the supernatant liquid as the solution comprising the oxidized product.

In step (ii) of the method of the invention, the oxidized product as produced is measured by a mass spectrometry technique. A mass spectrometry technique is known and available in this art which is an analytical technique that measures the mass-to-charge ratio of charged particles. In some embodiments, the mass spectrometry technique is a tandem mass spectrometry (MS/MS) technique, such as an LC-MS/MS technique. In general, in a MS analysis, the area under the peak is proportional to the amount of analyte injected onto the column. A peak's area can be determined by integration which is usually handled by the instrument's computer. If the peak is clear, the determination of the area under the peak is straightforward. As shown in the examples below, unique and distinct peaks with strong signal intensity are observed for the oxidized products of the compounds of the present invention.

In step (iii) of the method of the invention, a level of ceruloplasmin activity is determined based on the level (amount) of the oxidized product as measured through the mass spectrometry technique. According to the present invention, the level (amount) of the oxidized product as measured is positively correlated with the level of ceruloplasmin activity. Therefore, a decreased level (amount) of the oxidized product is indicative of a decreased level of ceruloplasmin activity and/or an increased level (amount) of the oxidized product is indicative of an increased level of ceruloplasmin activity. Particularly, the level (amount) of the oxidized product as measured can be further divided by the volume of the biological sample to represent a specific enzymatic activity unit. As shown in the examples below, the specific ceruloplasmin activity unit can be calculated by the formula: Specific Cp activity unit=Peak area of oxidized product MS2 ion/amount of serum (μL).

As used herein, a decreased level or increased level can refer to a level that is decreased or increased compared with a reference level. For example, a decreased level can be lower than a reference level by more than 10%, 20%, 30%, 40%, 50%, 60%, 70%, 80%, 90% or 100%; and an increased level can be higher than a reference level by more than 10%, 20%, 30%, 40%, 50%, 60%, 70%, 80%, 90% or 100%. In some embodiments, a reference level can be a standard (or a threshold) level in a control group. For example, a standard level can be set based on an average or median level obtained from a cohort of normal subjects. In particular embodiments, the cohort of subjects can be a population of normal human.

In some embodiment, the biological sample used in the present invention is a liquid biological sample, which includes but is not limited to a serum sample or a urine sample.

The method of the present invention provides excellent reliability and sensitivity. More specifically, the method of the present invention requires trace amounts of a biological sample to be tested and achieves desired reliability and sensitivity. In some embodiments, the method of the present invention requires the biological sample having a volume of 0.01 μL to 10 μL, for example, 0.05 μL to 5 μL, such as 0.05 μL to 2 μL, 0.05 μL to 1 μL or 0.05 μL to 0.5 μL.

Since ceruloplasmin activity is known to be associated with a number of diseases/conditions, the method of the present invention of determining ceruloplasmin activity is applicable in clinical applications for predicting the risk of a disease related to ceruloplasmin activity in a subject.

Therefore, the present invention also provides a method for determining ceruloplasmin activity and predicting the risk of a disease related to ceruloplasmin activity in a subject, comprising
(i) providing a biological sample which is obtained from the subject;
(ii) contacting the biological sample with a compound of formula I as described herein under a condition such that the compound is oxidized to produce an oxidized product of the compound;
(iii) measuring a level of the oxidized product by a mass spectrometry technique; and
(iv) determining a level of ceruloplasmin activity and the likelihood of having a disease or condition related to ceruloplasmin activity of said subject based on the level of the oxidized product, wherein
a decreased level of the oxidized product below a reference level is indicative of deficiency of ceruloplasmin activity and an increased likelihood of having a disease or condition related to deficiency of ceruloplasmin activity; and
an increased level of the oxidized product above a reference level is indicative of excess of ceruloplasmin activity and an increased likelihood of having a disease or condition related to excess of ceruloplasmin activity.

Some diseases or conditions are known to be corelated with deficiency of ceruloplasmin activity. For example, aceruloplasminemia exhibits deficiency of ceruloplasmin activity which is directly caused by Cp protein gene²⁰. Some neurodegenerative diseases, including Alzheimer's disease²⁶ and Parkinson's disease²⁷⁻²⁸ are known to exhibit deficiency of ceruloplasmin activity. Additional disease or condition related to deficiency of ceruloplasmin activity include Wilson's disease²¹ and Menke's disease.

Some diseases or conditions are known to be corelated with excess of ceruloplasmin activity. Examples of such diseases or conditions include Type II diabetes mellitus²², cardiovascular diseases, such as valvular heart disease²⁴ and coronary heart disease²⁵, bladder cancer,²⁹ chronic obstructive pulmonary disease³⁰ and kidney diseases.²³

In some embodiments, the method of the present invention is applicable for a fetus, a neonate, a child, an adolescent or an adult. In particular, the method of the present invention is applicable for a fetus, a neonate or a child where blood is difficult to draw.

The present invention also provides a kit for performing the method as described herein, which comprises the compound of formula I as the enzymatic substrate, a reaction buffer for oxidation, a quenching agent for stopping oxidation, and instructions for using the kit to determine ceruloplasmin activity and/or predicting the risk of a disease related to ceruloplasmin activity in a subject by using a mass spectrometry technique. The components including the compound of formula I, the reaction buffer and the quenching agent can be packaged in the form of a kit. For example, the components can respectively be packaged in separate containers or some of the components packed in one container and the other packed in another container.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

A liquid chromatography-tandem mass spectrometry (LC-MS/MS) method was developed and validated for the determination of serum oxidase activity. Through the combinatorial library screening of in-house compounds, including natural products, synthetic compounds, and heterocyclic molecules, hit substrates which provided unique and selective oxidized products in ESI-MS analysis were selected. To optimized the detection method, the modification of hit substrates and the alternation of assay conditions were applied. Furthermore, the lead substrates provided good quantitative measurement of serum oxidases activity with less amount of serum samples (<5 μL) in LC-MS/MS. At last, spike-in ceruloplasmin showed good linearity (R²=0.9971), representing that serum oxidases had positive correlation with ceruloplasmin as well as the modified substrates could be oxidized by both ceruloplasmin and serum oxidases. This method can be widely applied to the detection of disease related to serum oxidase activity.

### 1. Material and Methods

### 1.1 General Information

All reagents and solvents were purchased from commercial suppliers and used without further purification. All non-aqueous reactions were performed in oven-dried glassware under a slight positive pressure of argon unless otherwise noted. NMR spectra were recorded on dilute solutions in CDCl₃ and CD₃OD on Bruker AVANCE 600 spectrometer at ambient temperature. Chemical shifts are given in δ values and coupling constants J are given in Hz. The splitting patterns are reported as s (singlet), d (doublet), t (triplet), q (quartet), m (multiplet), and dd (double of doublets). Reactions were magnetically stirred and monitored by thin-layer chromatography on silica gel. Flash column chromatography was performed on silica gel of 40-63 μm particle size. Preparative TLC chromatography was performed on silica gel F254 preparative TLC plates (20×20 cm, 0.5 mm layer and fluorescence at 254 nm, Merck). Rotary evaporation was used to concentrate the reaction mixture.

### 1.2 Screening for Candidate Chemicals

The chemical library dissolved in DMSO individually to give stock concentrations of 1 mg/mL. Each chemical was placed in individual well and incubated with serum sample in acetate buffer (0.1M, pH 5.0) at 37° C. for 1 h. Additional DMSO was provided to inhibit oxidase activity. High-resolution ESI mass spectra (Bruker Daltonics spectrometer) was utilized to monitor the oxidized products.

### 1.3 Synthesis of Compounds as CP Substrates

### 1.3.1 Serotonin (or 5-Hydroxytryptamine) (ACK-1000)

Serotonin (or 5-Hydroxytryptamine), named as ACK-1000 herein, was purchased from Cayman Chemical Company (Ann Arbor, MI, U.S.A.).

### 1.3.2 Amino Carbazoles (ACK-1001 to ACK-1006)

### 1.3.2.1 General Procedure for 9-Alkyl-9H-Carbazole (Compound 2)

Carbazole (Compound 1) (500 mg, 3 mmol) was dissolved in dried DMF (4 mL), and sodium hydride (60%) (300 mg, 7.4 mmol) was added proportionally at 0° C. After vigorously stirring for 30 min, each alkyl compound (6 mmol) as an alkylating reagent was prepared in DMF (1 mL) and added dropwise to the reaction at 0° C. The mixture was stirred for 4 hr then the reaction was quenched by water (10 mL). The precipitate was dissolved by ethyl acetate (30 mL) and washed with brine. The organic layers were collected, dried with MgSO₄, and concentrated. The crude product was purified by CC and characterized by NMR and mass spectrometry.

### (i) 9-buyl-9H-carbazole

1-Bromobutane (652 μL, 6 mmol) was used as the alkyl compound. The crude product was purified by CC (10% EA in hexanes, silica gel) to give N-butyl carbazole (647 mg, 2.9 mmol, 98% yield) as a white solid. ¹H NMR (600 MHz, CDCl₃) δ 3.89 (t, J=4.8 Hz, 3H), 7.25-7.29 (m, 2H), 7.42-7.45 (m, 2H), 7.49-7.53 (m, 2H), 8.12-8.15 (m, 2H).

### (ii) 9-methyl-9H-carbazole

Iodomethane (374 μL, 6 mmol) was used as the alkyl compound. The crude product was purified by CC (20% EA in hexanes, silica gel) to give N-methyl carbazole (436 mg, 2.4 mmol, 80% yield) as a white solid.¹H NMR (600 MHz, CDCl₃) δ 3.89 (t, 3H, J=4.1 Hz), 7.25-7.29 (m, 2H), 7.42-7.45 (m, 2H), 7.49-7.53 (m, 2H), 8.12-8.15 (m, 2H).

### (iii) 2-(9H-carbazol-9-yl)ethan-1-ol

3-Bromoethanol (440 μL, 6 mmol) was used as the alkyl compound. The crude product was purified by CC (40% EA in hexanes, silica gel) to give N-(2-hydroxyl)ethyl carbazole (506 mg, 2.4 mmol, 40% yield) as a white solid. ¹H NMR (600 MHz, CDCl₃) δ 4.06 (dt, J=5.3, 5.5 Hz, 2H), 4.48 (t, J=5.5 Hz, 2H), 7.23-7.25 (m, 2H), 7.46-7.47 (m, 4H), 8.09-8.10 (m, 2H)

### (iv) 3-(9H-carbazol-9-yl)propan-1-ol

3-Bromopropanol (545 μL, 6 mmol) was used as the alkyl compound. Furthermore, the reaction mixture was microwaved for 1 hr at 135° C. The crude product was purified by CC (40% EA in hexanes, silica gel) to give N-(3-hydroxyl)propyl carbazole (715 mg, 3.2 mmol, 53% yield) as a white solid. ¹H NMR (600 MHz, CDCl₃) δ 2.13 (quin, J=6.0 Hz, 2H), 3.62 (dt, J=5.6, 3.9 Hz, 2H), 4.48 (t, J=6.7 Hz, 2H), 7.22-7.25 (m, 2H), 7.46-7.47 (m, 2H), 8.10-8.11 (m, 2H).

### (v) 9-(2,5,8,11-tetraoxatridecan-13-yl)-9H-carbazole

Tetraethylene glycol (288 mg, 1.48 mmol) and potassium hydroxide (99 mg, 1.77 mmol) were added to the mixed solvent of water (1 mL) and 1,4-dioxane (4 mL). After stirring for 2 min, dimethylsulfate (50 μL, 0.47 mmol) was added dropwise to the reaction at room temperature. Subsequently, the mixture was heated by microwave at 135° C. for 1 hour. To the end of the reaction, the solvent was evaporated and water (30 mL) was added in. The solution was extracted by DCM and washed with brine. The combination of organic layers were dried with MgSO₄ and concentrated. The crude product (100 mg, 0.48 mmol) and triethylamine (88 μL, 0.62 mmol) were added to dried DCM under ice bath. Tosylchloride (110 mg, 0.57 mmol) prepared in DCM (1 mL) was added dropwise to the solution. After 2 hours of stirring, the solvent was evaporated and re-dissolved with EA. The organic layers were washed with brine, dried with MgSO₄, and concentrated. The crude product was purified by CC (DCM: methanol=19:1) to give 2,5,8,11-tetraoxatridecan-13-yl 4-methylbenzenesulfonate (133 mg, 0.4 mmol, 85% yield).

2,5,8,11-Tetraoxatridecan-13-yl 4-methylbenzenesulfonate (323 mg, 0.9 mmol) was used as the alkyl compound. The crude product was purified by CC (5% methanol in DCM, silica gel) to give the N-alkylated carbazole (80 mg, 0.22 mmol, 37% yield) as a colorless oil. ¹H NMR (600 MHz, CDCl₃) δ 3.15 (s, 3H), 3.28-3.36 (m, 14H), 3.64 (t, J=6.0 Hz, 2H), 4.27 (t, J=6.1 Hz, 2H), 7.01-7.03 (m, 2H), 7.24 (m, 4H), 7.86-7.88 (m, 2H).

### (vi) 9-ethyl-9H-carbazole

1-Bromoethane (445 μL, 6 mmol) was used as the alkyl compound. The crude product was purified by CC (10% EA in hexanes, silica gel) to give N-butyl carbazole (556 mg, 2.9 mmol, 95% yield) as a white solid. ¹H NMR (600 MHz, CDCl₃) δ 1.40 (t, 3H, J=7.2 Hz), 4.31 (q, 2H, J=7.2 Hz), 7.25-7.29 (m, 2H), 7.42-7.45 (m, 2H), 7.49-7.53 (m, 2H), 8.12-8.15 (m, 2H).

1.3.2.2 General Procedure for 9-alkyl-9H-carbazol-3-amine (Compound 3)

Compound 2 (100 mg, 0.45 mmol) was dissolved in acetic acid (3 mL), and stirred for 5 min under room temperature. A dissolved Cu(NO₃)₂ solution (containing Cu(NO₃)₂·3H₂O (140 mg, 0.60 mmol), acetic anhydride (0.2 mL) and acetic acid (1 mL)) was added dropwise to the carbazole solution. After stirring for 1 hr, water was added to the solution and the pH value was adjusted to 7-8 with 10% NaOH_{(aq)}. Subsequently, the mixture was extracted by EA (30 mL, three times) and washed with brine. The combined organic layers were dried with MgSO₄ and concentrated. The crude product was purified by CC to afford nitrated compounds. The nitrated compounds (100 mg, 0.37 mmol) was dissolved in THF/methanol (2 mL, 2 mL) then Pd/C (1 mg) was added to the solution. The mixture was stirred at 60° C. for 2 min and the solution of hydrazine monohydrate (600 μL in 1 mL methanol) was added dropwise to the mixture. After the reaction was checked by TLC, the solvent of the reaction was evaporated and the residue was dissolved by EA (45 mL). The organic layers were washed with brine, dried with MgSO₄ and concentrated. The crude product was purified by CC and characterized by NMR and mass spectrometry.

### (i) 9-buyl-9H-carbazol-3-amine (ACK-1001)

The crude product was purified by preparative TLC (50% DCM and 1% NH₄OH in hexanes) to give ACK-1001 (22 mg, 0.09 mmol, 25% yield) as a dark red solid. ¹H NMR (600 MHz, CDC₃), δ=0.93 (t, J=7.3 Hz, 3H), 1.38 (sext, J=7.7 Hz, 2H), 1.82 (quint, J=7.5 Hz), 4.24 (t, J=7.2 Hz), 6.91 (dd, J=1.7, 8.6 Hz, 1H), 7.14 (t, J=7.5 Hz, 1H), 7.21 (d, J=8.4 Hz, 1H), 7.33 (d, J=8.1 Hz, 1H), 7.39-7.42 (m, 2H), 7.99 (d, J=7.6 Hz, 1H).

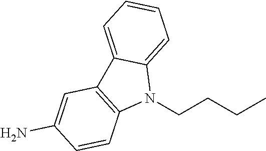 [SMILES: CCCCn1c2ccccc2c2cc(N)ccc21]

### (ii) 9-methyl-9H-carbazol-3-amine (ACK-1002)

The crude product was purified by CC (50% DCM and 1% NH₄OH in hexanes) to give ACK-1002 (36 mg, 0.18 mmol, 40% yield) as a dark red oil. ¹H NMR (600 MHz, CDCl₃), δ=3.79 (s, 3H), 6.92 (dd, J=1.5, 8.3 Hz, 1H), 7.15 (t, J=7.4 Hz, 1H), 7.21 (d, J=8.2 Hz, 1H), 7.33 (d, J=8.2 Hz, 1H), 7.41-7.44 (m, 2H), 7.99 (d, J=7.7 Hz, 1H).

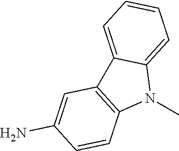

### (iii) 2-(3-amino-9H-carbazol-9-yl)ethan-1-ol (ACK-1003)

The crude product was purified by CC (5% methanol and 1% NH₄OH in DCM) to give ACK-1003 (7 mg, 0.03 mmol, 8% yield) as a dark red oil. ¹H NMR (600 MHz, CDCl₃), δ=4.03 (t, J=5.3 Hz, 2H), 4.42 (t, J=5.3 Hz, 2H), 6.90 (dd, J=2.2, 8.5 Hz, 1H), 7.17 (m, 1H), 7.27 (m, 1H), 7.40-7.41 (m, 3H), 7.89 (dd, J=3.3, 7.5 Hz, 1H).

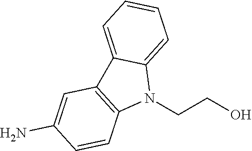 [SMILES: Nc1ccc2c(c1)c1ccccc1n2CCO]

### (iv) 3-(3-amino-9H-carbazol-9-yl)propan-1-ol (ACK-1004)

The crude product was purified by CC (5% methanol and 1% NH₄OH in DCM) to give ACK-1004 (13 mg, 0.05 mmol, 27% yield) as a dark red solid. ¹H NMR (600 MHz, CDCl₃), δ=2.09 (quint, J=6.0 Hz, 2H), 3.61 (t, J=6.5 Hz, 2H), 4.40 (dt, J=6.4, 6.6 Hz, 2H), 6.97 (dd, J=7.7, 7.7 Hz, 1H), 7.08 (dd, J=7.7, 7.7 Hz, 1H), 7.20 (dd, J=6.8, 7.4 Hz, 1H), 0.7.27 (s, 1H), 7.40-7.44 (m, 2H), 8.23 (t, J=7.44 Hz, 1H).

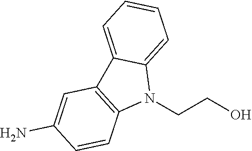 [SMILES: Nc1ccc2c(c1)c1ccccc1n2CCO]

### (v) 9-(2,5,8,11-tetraoxatridecan-13-yl)-9H-carbazol-3-amine (ACK-1005)

The crude product was purified by CC (5% methanol and 1% NH₄OH in DCM) to give ACK-1005 (5 mg, 0.01 mmol, 26% yield) as a dark red oil. ¹H NMR (600 MHz, CDCl₃), δ=3.35 (s, 3H), 3.50-3.55 (m, 10H), 3.58-3.59 (m, 2H), 3.83 (t, J=6.5 Hz, 2H), 4.43 (t, J=6.4 Hz, 2H), 6.89 (dd, J=2.4, 8.6 Hz, 1H), 7.14 (ddd, J=2.1, 5.8, 7.4 Hz, 1H), 7.27 (d, J=8.5 Hz, 1H), 7.39-7.40 (m, 3H), 7.96 (m, 1H).

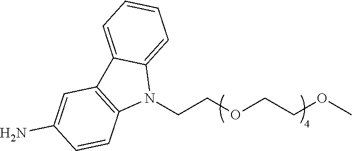

### (vi) 9-ethyl-9H-carbazol-3-amine (ACK-1006)

The crude product was purified by CC (50% DCM and 1% NH₄OH in hexanes) to give ACK-1006 (70 mg, 0.33 mmol, 90% yield) as a dark red oil. ¹H NMR (600 MHz, CDCl₃), δ=1.40 (t, 3H, J=7.2 Hz), 4.31 (q, 2H, J=7.2 Hz), 6.91 (dd, J=2.1, 6.42 Hz, 1H), 7.15 (t, J=7.4 Hz, 1H), 7.21 (d, J=8.5 Hz, 1H), 7.33 (d, J=8.1 Hz, 1H), 7.41-7.44 (m, 2H), 7.99 (d, J=7.7 Hz, 1H).

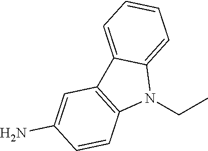 [SMILES: CCn1c2ccccc2c2cc(N)ccc21]

### 1.4 LC-MS/MS Based Serum Oxidase Activity Assay

Samples were detected by LC-ESI-MS on a Velos Pro dual-pressure linear ion trap mass spectrometer (Thermo Fisher Scientific, San Jose, CA) equipped with an Ultimate 3000 RSLC system from Dionex (Dionex Corporation, Sunnyvale, CA), using a Xbridge C18 (HPLC column 1 mm×15 cm i.d., 3.5 μm particle size, 130 Å pore size). Briefly, the gradient employed was 50% buffer B at 5 min to 98% buffer B at 23 min with a flow rate of 50 μL/min, where buffer A was 0.1% formic acid/H₂O and buffer B was 0.1% formic acid/acetonitrile. All spectra were acquired in the mass range m/z 200-2000. Electrospray voltage was maintained at 4.0 kV and capillary temperature was set at 275° C.

The stock solution of modified amino carbazoles (125 μM, 20 μL) were incubated with 5 μL of serum (estimate<10 nM of serum oxidases) in sodium acetate buffer (0.1M, pH 5.0, 5 μL) at 37° C. for 30 min. The serum oxidase oxidation was quenched by DMSO (90 μL), followed detection of LC-MS/MS.

The amount of the oxidized product was determined according to the area under the peak by integration. The specific Cp activity unit was calculated by the formula: Peak area of oxidized product MS2 ion/serum volume (μL).

### 2. Results

### 2.1 Compounds as Substrates for Serum (ACK-1000 and ACK-1001 to ACK-1006)

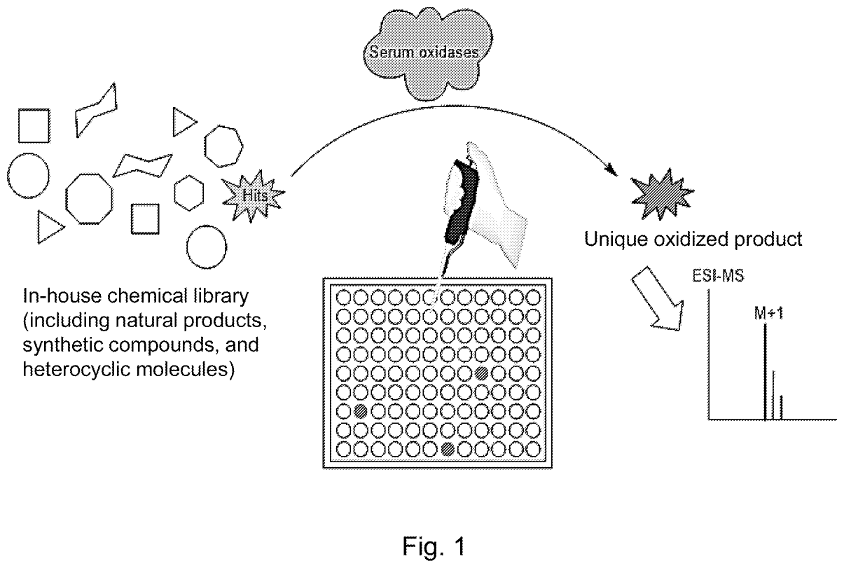

To screen for suitable compounds as Cp substrates for MS analysis, an approach of combinatorial library screening in primary MS analysis was used to find out oxidized products and their relationship with initial substrates. Briefly, a platform for rapidly and conveniently screening various small molecules was established. In the chemical library collection as obtained, including natural products, synthetic compounds and heterocyclic molecules, each member was parallelly incubated with a certain small amount of serum for a desired reaction time. After quenching each enzyme reaction, analysis of the reaction products with ESI-MS was performed. Hits with reliable, major, and distinct oxidized product peaks in ESI-MS analysis were selected for further studies (FIG. 1).

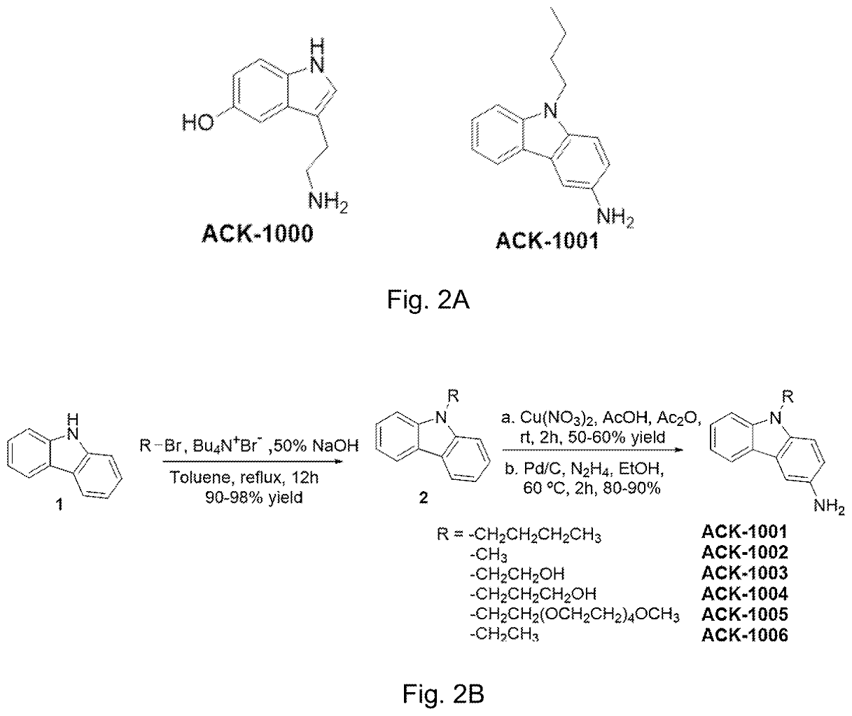

Through screening, certain heterocyclic compounds, ACK-1000 and ACK-1001 (FIG. 2A) were sieved out as candidates since these compounds were found to generate specific oxidized products instead of unclear polymeric mixtures when reacting with serum oxidases. Further, chemical modifications were performed to obtain a series of modified amino carbazole derivatives, including ACK-1002, ACK-1003 ACK-1004, ACK-1005, ACK-1006 (FIG. 2B). As shown in FIG. 2B, Compound 1 was first alkylated by different alkyl chains to give alkylated carbazole 2 followed by the nitration at the 3rd position. Furthermore, the reduction of the nitration products with Pd/H₂NNH₂ gave higher yields of all the reduction conditions we had tried. Finally, a series of modified amino carbazole derivatives were synthesized.

### 2.2 LC-MS/MS Detection of Serum Oxidase Activity

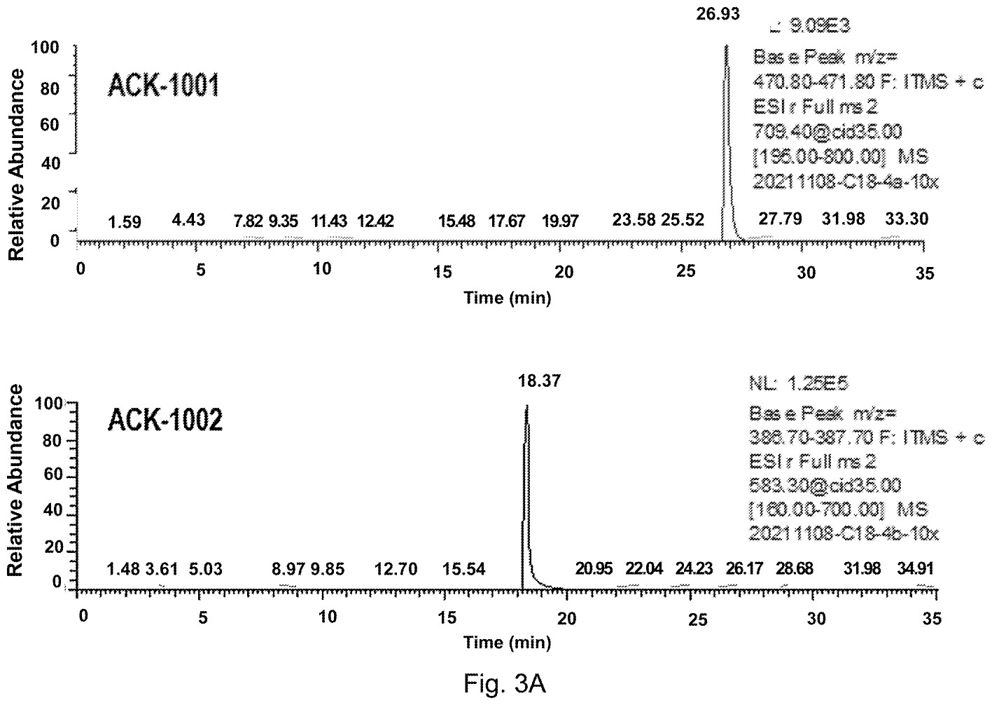

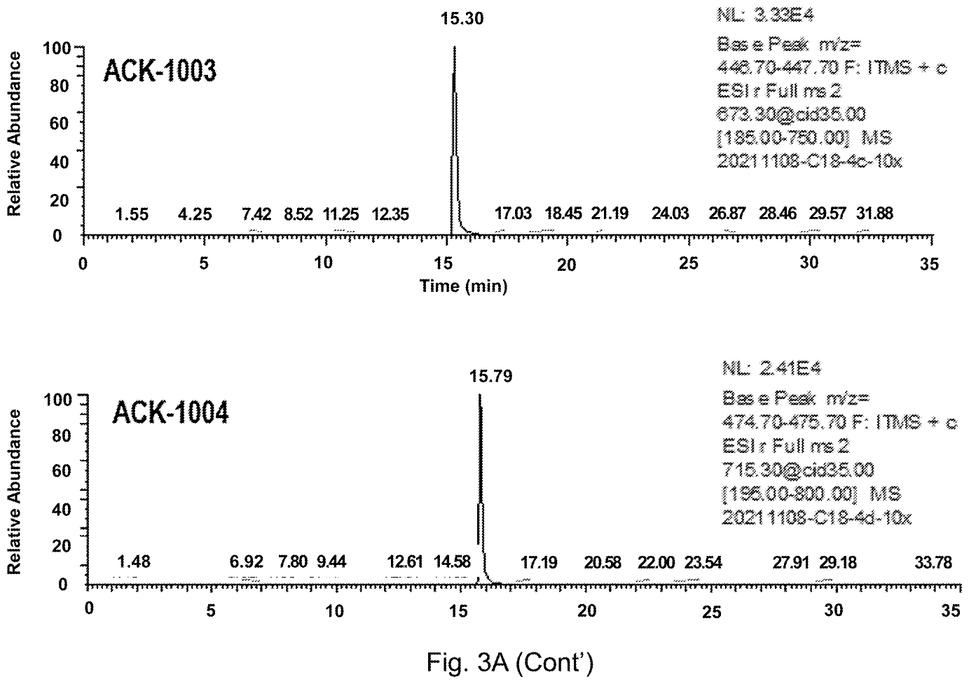

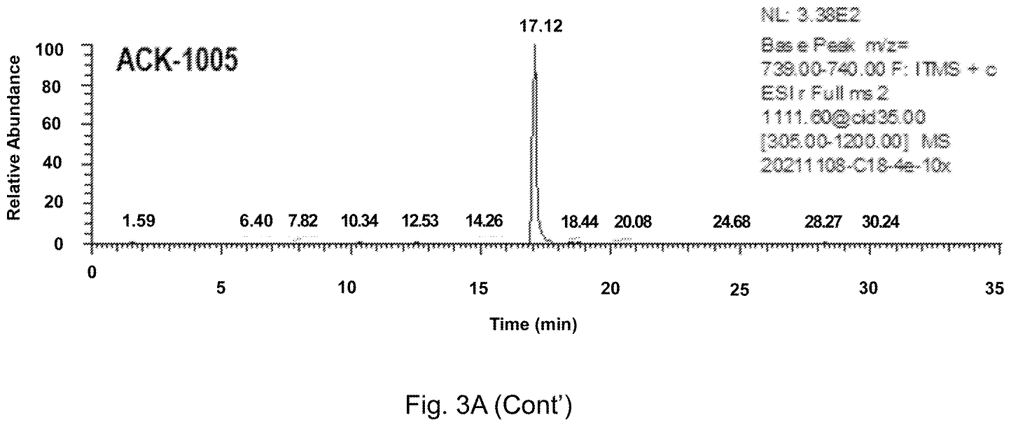

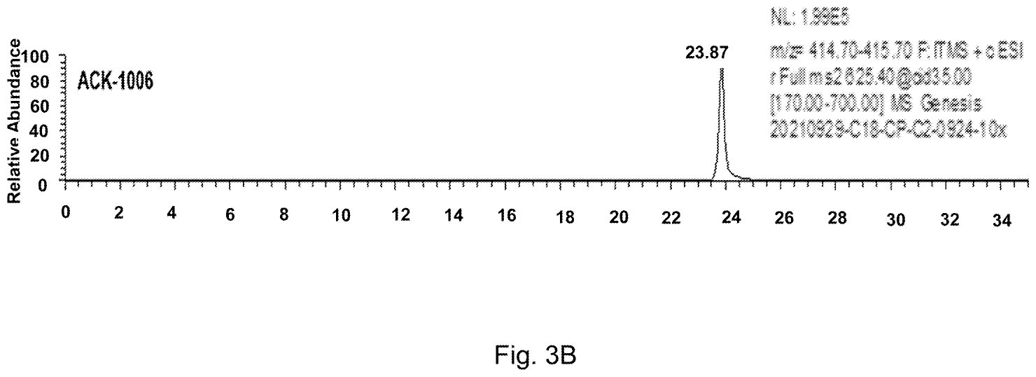

Detection of serum oxidase activity with the candidate compounds was performed by LC-MS/MS. Unique and distinct peaks with strong signal intensity were observed, indicating that the oxidized products of these candidate compounds (ACK-1001 to ACK-1006) are reliable bio-markers for detection of serum oxidases (FIG. 3A and FIG. 3B).

2.3 Substrates with Specific Reactivity for CP

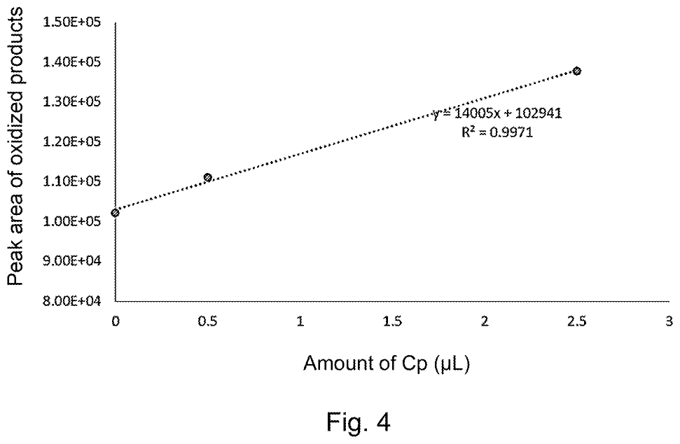

To understand the relation between ceruloplasmin and serum oxidases and the reactivity of modified substrates and ceruloplasmin, spike-in experiment was performed. Different amounts of recombinant ceruloplasmin (0, 0.5, and 2.5 μL) were added in the normal serum sample (5 μL) and reacted with ACK-1006. The assay was incubated at 37° C. for 30 mins, and quenching the reaction by DMSO. The final products were analyzed by LC-MS/MS. By comparing the LC peak area, the increasing amount of ceruloplasmin contributed the growing quantity of oxidized product and showed good linearity (R²=0.9971) (FIG. 4). Spike-in recombinant ceruloplasmin showed that serum oxidases had positive correlation with ceruloplasmin concentrations as well as the modified substrates ACK-1006 could be oxidized by recombinant ceruloplasmin.

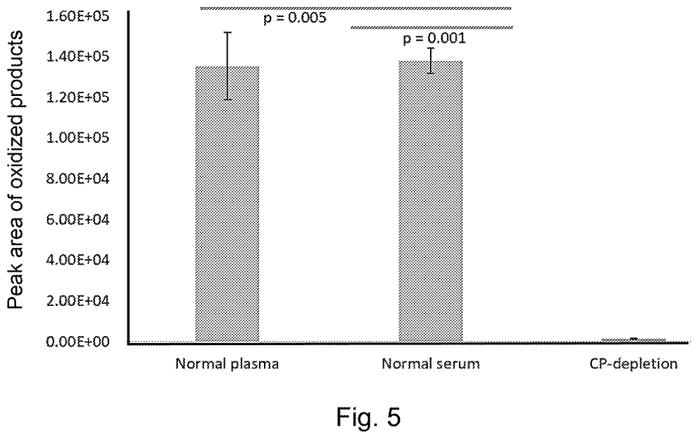

Another way to confirm that the ACK-1006 substrate exclusively reacts with ceruloplasmin, protein depletion test was applied. The Cp depletion serum was obtained by size selection method. Briefly, the centrifuge filter was applied in normal serum to eliminate those proteins larger than 100 kDa, including Cp (˜130 kDa). ACK-1006 was incubated with the Cp depleted serum and trace amount of oxidized product were detected by LCMS. Comparison with serum and plasma from normal adults representing p value smaller than 0.001 indicating that ACK-1006 is affected by Cp activity (FIG. 5).

### 2.4 Establishment of LC-MS/MS Based Measurement of Serum Cp Oxidase Activity

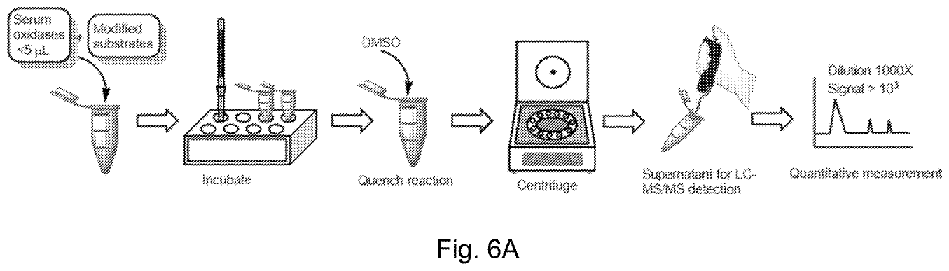

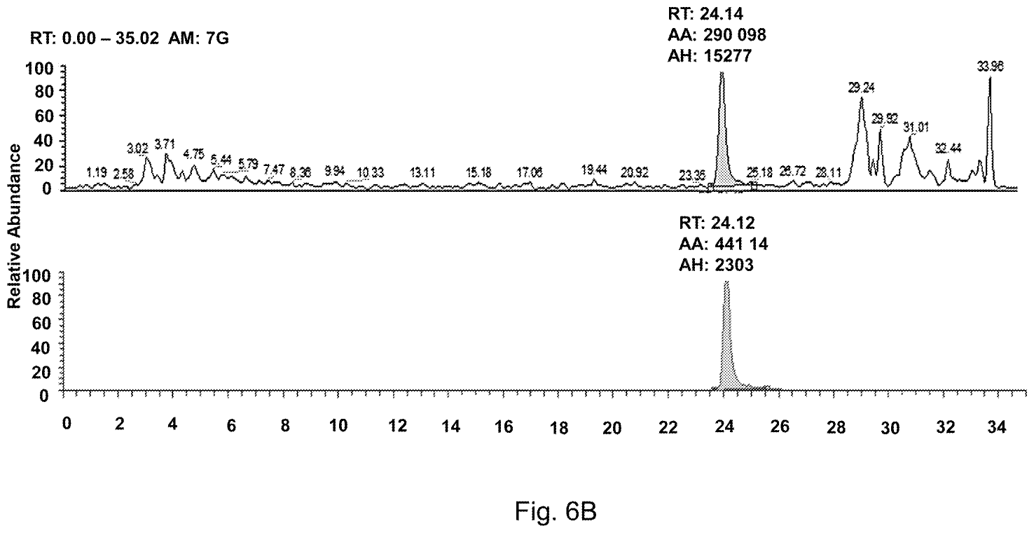

Accordingly, the method of LC-MS/MS based assay based on the determination of the oxidized products was established (FIG. 6A). As shown in FIG. 6A, serum samples, less than 5 μL in each analytical test, were incubated with a synthetic substrate (ACK-1006 as an example) in sodium acetate buffer. The addition of DMSO was to quench the serum enzyme activity. After centrifugation, the supernatant of the mixture was detected by LC-MS/MS. After proper dilution, for instance, making a 1000-fold dilution, the qualified mass/charge (m/z) transition of the corresponding oxidized product with signal intensity>10³ was observed, indicating that the new Cp assay substrate with LC-MS/MS method provided a great sensitivity and the substrate and the corresponding oxidized product were qualified as selective and stable biomarkers (FIG. 6B). This result can provide precise and quantitative measurement of serum Cp oxidase activity.

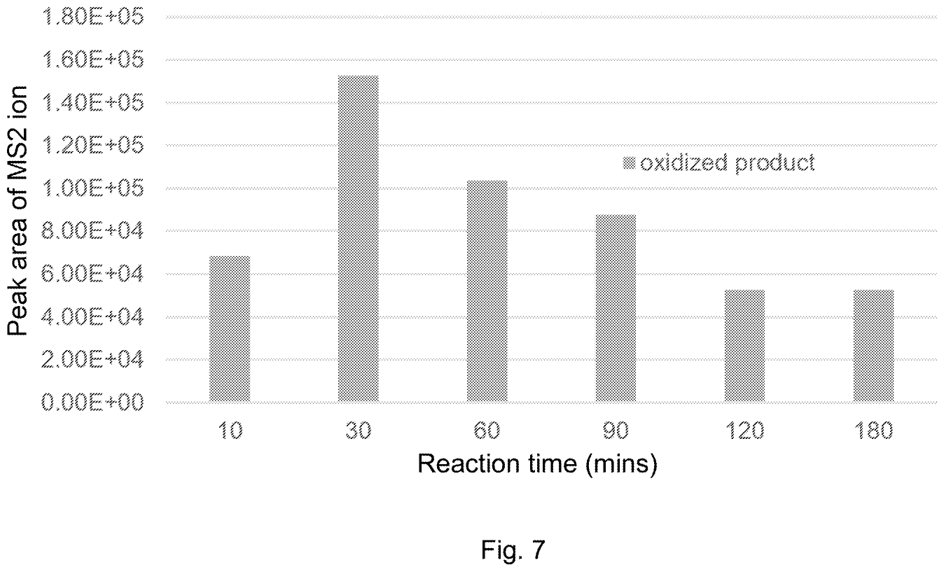

To obtain the accurate and precise analysis, assay conditions have been further optimized. Substrate ACK-1006 was used in the test and detection was taken at different reaction times. The time-dependent assay exhibited that oxidized product reached highest amount after 30 min reaction, indicating that serum Cp oxidase is active within 30 min incubation. Thus, the assay time has been adjusted to accomplish within 30 mins (FIG. 7).

### 2.5 Limitations of Detection

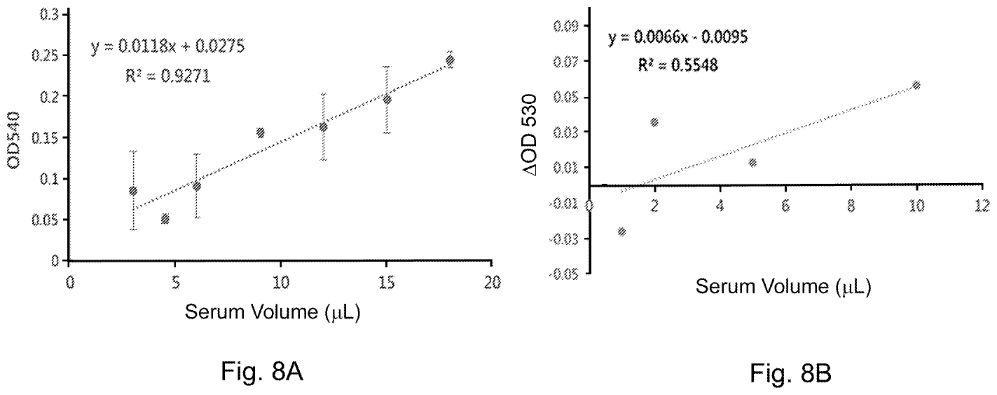

The limitations of current methods and available commercial kits were described. In current method, the colorimetric methods using p-phenylenediamine (PPD) and o-dianisidine dihydrochloride (ODD) as substrates are used for quantification of ceruloplasmin oxidative activity. Those substrates oxidized by ceruloplasmin and resulted in obvious color changes. The activity of ceruloplasmin were positively correlated to absorbance. However, the correlation of absorbance and serum volume small than 10 μL was poor (FIG. 8).

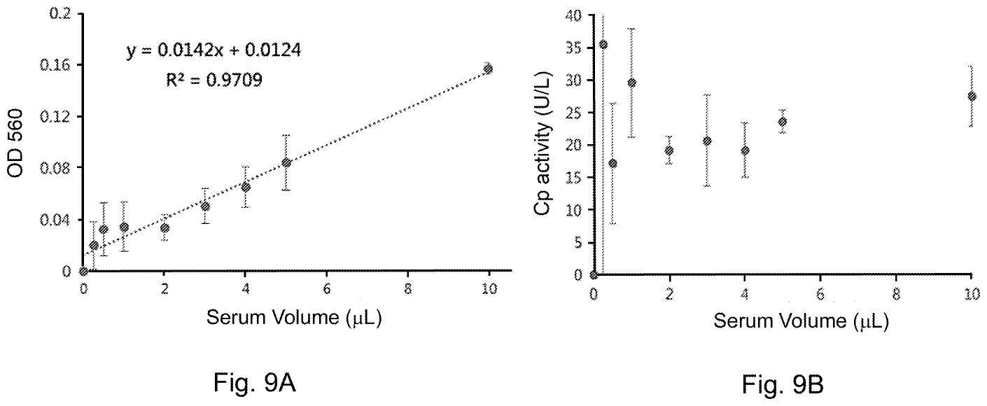

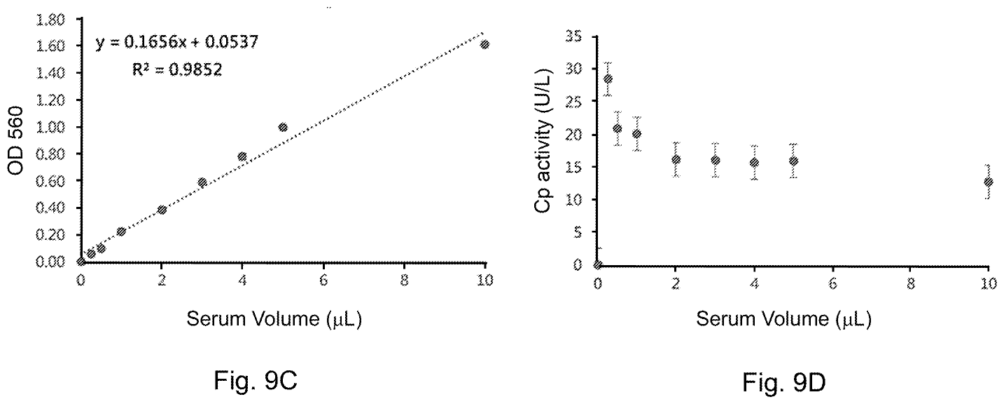

Two commercial kits were available (BioVision kit and Invitrogen kit) for quantification of serum Cp activity. There was a good correlation between absorbance and serum volume from 0.25 μL to 10 μL. (FIGS. 9A and 9C). The Cp activity unit (Cp units/serum volume) was stable from the range of 2 μL to 10 μL serum. However, the Cp activity unit was not reliable under 2 μL of serum (FIGS. 9B and 9D).

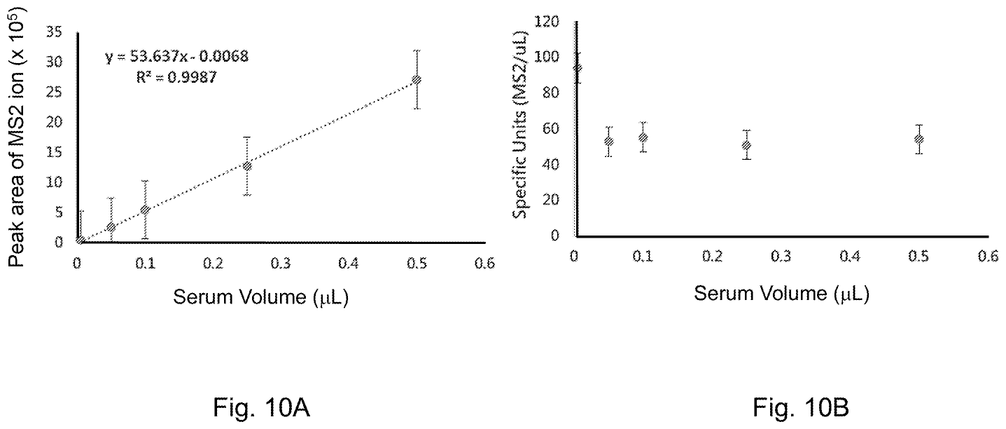

To detect the limitation of ACK1006-LC/MS method, the ACK-1006 and serum oxidation mixture were diluted to 10-1,000 folds and analyzed by LC-MS/MS. The calibration curve exhibited a good correlation between the observed peak area of MS2 ion (y) and the serum volume (x) (R²=0.9987) (FIG. 10A). The Cp activity unit can still be detected under extremely lower serum volume conditions (0.05 μL to 0.5 μL) (FIG. 10B). These results indicated that current absorbance-based methods were unreliable in applications for those sample with lower serum volume or lower Cp activity, whereas the ACK1006-LC/MS method provides remarkable detection limitation. For example, dried blood spots (DBS) were mostly used to detect metabolites for newborn screening (NBS), however, the content of serum is extremely low. Furthermore, it has been reported that the amounts of Cp protein in newborn is much lower than adults. Hence, the ACK1006-LC/MS method for quantification of Cp activity is potentially reliable in newborn screening for Cp related severe diseases, such as Menkes' syndrome, Wilson's disease, Overdose of Vitamin C and Copper deficiency.

### 2.6 Detection of Cp Activity in Newborn Serum

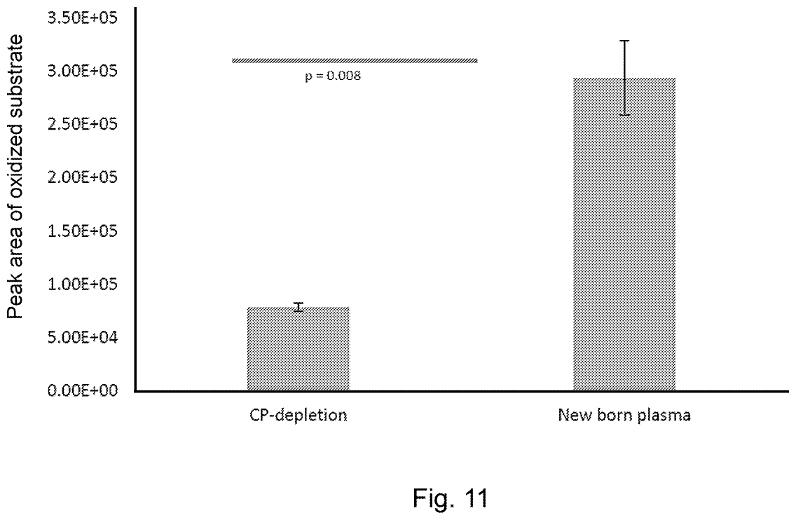

This established Cp-activity assay by novel substrate ACK-1006 in LC-MS was also reliable even in the newborn plasma which contains extreme lower ceruloplasmin than normal adults. Substrate ACK-1006 were oxidized by new born plasma and showed a distinctive signal (>10⁵) by LCMS (FIG. 11). The signals of oxidative ACK-1006 substrate were still significantly lower in CP-depletion plasma when compared with newborn plasma. This comforting result may prompt us to use this method to establish a novel assay for clinical applications for those diseases with abnormal amounts or oxidative activities of ceruloplasmin in newborns.

### 3. CONCLUSIONS

Through our efforts, ACK-1000 was initially found out via diverse library screening with the LC-MS analysis. After structural modifications, a series of analogues (ACK1001-1006) were designed and synthesized for further investigation. Fortunately, qualified new Cp substrates were identified with an assistance of LC-MS/MS detection. The relative standard deviation (<12%) exhibited excellent stability of MS spectrometry, and a squares regression R²=0.9968 displayed that the LC-MS/MS based assay provided decent linearity and sensitivity. In addition, spike-in recombinant ceruloplasmin showed that the modified substrate ACK-1006 as an example had a positive correlation with ceruloplasmin amounts. In contrast, the incubation of Cp-depletion serum and ACK-1006 produced trace amount of an oxidized signal from the oxidation of substrate in comparison with normal serum (P value=0.001). Both Cp spike-in and depletion experiment showed that ACK-1006 is specifically affected by Cp activity. Thus, the ACK1006-LC-MS/MS micro-analytical method is suitable for clinical applications of those diseases with abnormal amounts or oxidative activities of Cp in newborns.

### REFERENCES

1. G. Wu, Y. Z. Fang, S. Yang, J. R. Lupton, N. D. Turner, J. Nutr 2004, 134 (3), 489.
2. I. I. Vlasova, Molecules, 2018, 23 (10), 2561.
3. R. Squitti, M. Ventriglia, M. Siotto, C. Salustri, Biometals in Neurodegenerative Diseases (Eds.: A. R. White, M. Aschner, L. G. Costa, A. I. Bush), Academic Press, 2017, pp. 19-34.
4. H. P. Roeser, G. R. Lee, S. Nacht, G. E. Cartwright, J. Clin. Investig. 1970, 49 (12), 2408.
5. A. Kulkarni, V. K. Sharma, International Encyclopedia of Public Health (Second Edition) (Eds.: S. R. Quah), Academic Press, Oxford, 2017, pp. 424-433.
6. U. Merle, C. Eisenbach, K. H. Weiss, S. Tuma, W. Stremmel, J. Hepatol. 2009, 51 (5), 925.
7. N. E. Hellman, J. D. Gitlin, Annu. Rev. Nutr 2002, 22 (1), 439.
8. Z. L. Harris, Clinical and Translational Perspectives on WILSON DISEASE (Eds.: N. Kerkar, E. A. Roberts), Academic Press, 2019, pp. 77-84.
9. K. Hirano, T. Ogihara, H. Ogihara, M. Hiroi, M. Hasegawa, H. Tamai, Clin. Biochem. 2005, 38 (1), 9.
10. M. C. Linder, Metallomics, 2016, 8 (9), 887.
11. Y. Huang, P. Chan, G. Halliday, Oxidative Stress and Neurodegenerative Disoiders, Elsevier, 2007, pp. 663-697.
12. K. Strzelak, J. Glowacka, R. Koncki, Talanta, 2020, 214, 120881.
13. K. H. Schosinsky, H. P. Lehmann, M. F. Beeler, Clin. Chem. 1974, 20 (12), 1556.
14. K. M. Stepien, M. Guy, Ann. Clin. Biochem. 2018, 55 (1), 149.
15. G. Ćirić-Marjanović, M. Milojević-Rakić, A. Janosěvid-Ležaić, S. Luginbühl, P. Walde, Chem. Pap. 2017, 71 (2), 199.
16. B. J. Bythell, C. L. Hendrickson, A. G. Marshall, J. Am. Soc. Mass Spectom. 2012, 23 (4), 644.
17. M. Whitmire, J. Ammerman, P. Lisio, J. Anal. Bioanal. Tech. 2011, 01.
18. F. Gosetti, E. Mazzucco, D. Zampieri, M. C. Gennaro, J. Chromatogr A. 2010, 1217 (25), 3929.
19. X. Luo, N. F. Cai, Z. N. Cheng, J. Pharm. Biomed. Anal. 2013, 81-82, 8-12.
20. Vila Cuenca, M.; Marchi, G.; Barque, A.; Esteban-Jurado, C.; Marchetto, A.; Giorgetti, A.; Chelban, V.; Houlden, H.; Wood, N. W.; Piubelli, C.; Dorigatti Borges, M.; Martins de Albuquerque, D.; Yotsumoto Fertrin, K.; Jove-Buxeda, E.; Sanchez-Delgado, J.; Baena-Diez, N.; Burnyte, B.; Utkus, A.; Busti, F.; Kaubrys, G.; Suku, E.; Kowalczyk, K.; Karaszewski, B.; Porter, J. B.; Pollard, S.; Eleftheriou, P.; Bignell, P.; Girelli, D.; Sanchez, M., Genetic and Clinical Heterogeneity in Thirteen New Cases with Aceruloplasminemia. Atypical Anemia as a Clue for an Early Diagnosis. Int J Mol Sci 2020, 21 (7).
21. Kulkarni, A.; Sharma, V. K., Wilson's Disease. In International Encyclopedia of Public Health (Second Edition), Quah, S. R., Ed. Academic Press: Oxford, 2017; pp 424-433.
22. Raut, M.; Bhattarai, A.; Tuladhar, E. T.; Yadav, B. K.; Shrestha, S.; Aryal, B.; Pant, V.; Tumbapo, A.; Sharma, V. K., Ceruloplasmin, a potential marker for glycemic status and its relationship with lipid profile in Type II diabetes mellitus. Asian Journal of Medical Sciences 2018, 9 (2), 13-18.
23. Gudehithlu, K. P.; Hart, P.; Joshi, A.; Garcia-Gomez, I.; Cimbaluk, D. J.; Dunea, G.; Arruda, J. A. L.; Singh, A. K., Urine exosomal ceruloplasmin: a potential early biomarker of underlying kidney disease. Clin Exp Nephrol 2019, 23 (8), 1013-1021.
24. Sacheat, H. K.; Husseinand, S. Z.; Al-Mudhaffar, S. S., Evaluation of Ceruloplasmin ferroxidase activity and lipid profiles in patients with Valvular heart diseases. Journal of Physics: Conference Series 2021, 1879 (2), 022066.
25. Arenas de Larriva, A. P.; Limia-Perez, L.; Alcala-Diaz, J. F.; Alonso, A.; Lopez-Miranda, J.; Delgado-Lista, J., Ceruloplasmin and Coronary Heart Disease-A Systematic Review. Nutrients 2020, 12 (10).
26. Zhao, Y. S.; Zhang, L. H.; Yu, P. P.; Gou, Y. J.; Zhao, J.; You, L. H.; Wang, Z. Y.; Zheng, X.; Yan, L. J.; Yu, P.; Chang, Y. Z., Ceruloplasmin, a Potential Therapeutic Agent for Alzheimer's Disease. Antioxid Redox Signal 2018, 28 (14), 1323-1337.
27. Bakeberg, M. C.; Riley, M.; Byrnes, M.; Jefferson, A.; Ghosh, S.; Horne, M. K.; McGregor, S.; Stell, R.; Walters, S.; Evans, T.; Roberts, K.; Mastaglia, F. L.; Anderton, R. S., Elevated Serum Ceruloplasmin Levels Are Associated with Higher Impulsivity in People with Parkinson's Disease. Parkinsons Dis 2020, 2020, 8296203.
28. Grolez, G.; Moreau, C.; Sablonniere, B.; Garcon, G.; Devedjian, J. C.; Meguig, S.; Gele, P.; Delmaire, C.; Bordet, R.; Defebvre, L.; Cabantchik, I. Z.; Devos, D., Ceruloplasmin activity and iron chelation treatment of patients with Parkinson's disease. BMC Neurol 2015, 15, 74.
29. Mukae, Y.; Ito, H.; Miyata, Y.; Araki, K.; Matsuda, T.; Aibara, N.; Nakamura, Y.; Matsuo, T.; Sakai, H.; Ohyama, K., Ceruloplasmin Levels in Cancer Tissues and Urine Are Significant Biomarkers of Pathological Features and Outcome in Bladder Cancer. Anticancer Res 2021, 41 (8), 3815-3823.
30. Lalita V. Kamble, D. M. V. R. J. a. D. M. J. G., To study the level of Nitric Oxide and Ceruloplasmin in Chronic Obstructive Pulmonary Disease. International Journal of Biotechnology & Biochemistry (IJBB) 2020, 16 (1), 45-49.

The invention claimed is:

1. A method of determining ceruloplasmin activity, comprising:
(i) contacting a biological sample containing ceruloplasmin with a compound of formula I under a condition such that the compound reacts with the ceruloplasmin to produce an oxidized product of the compound 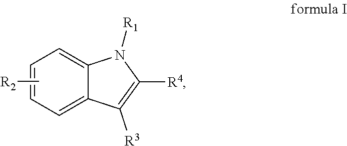
wherein
R₁ is hydrogen, an alkyl group, an alcohol group, an alkoxy group or an alkyl ether group containing one or more oxyethylene units;
R₂ is hydrogen or hydroxyl; and
R₃ and R₄, the same or different, are independently hydrogen or an alkylamine group, or
R₃ and R₄ together form a benzene ring fused to the nitrogen-containing bicyclic heteroring, wherein the benzene ring is substituted by —NH₂;
(ii) measuring a level of the oxidized product by a mass spectrometry technique; and
(iii) determining a level of ceruloplasmin activity based on the level of the oxidized product, wherein a decreased level of the oxidized product is indicative of a decreased level of ceruloplasmin activity, and an increased level of the oxidized product is indicative of an increased level of ceruloplasmin activity.

2. The method of claim 1, wherein
R₁ is hydrogen, an alkyl group, an alcohol group, an alkoxy group or an alkyl ether group containing one or more oxyethylene units;
R₂ is hydrogen or hydroxyl; and
R₃ and R₄, the same or different, are independently hydrogen or an alkylamine group.

3. The method of claim 2, wherein
R₁ is hydrogen;
R₂ is hydroxyl;
R₃ is an alkylamine group;
R₄ is hydrogen.

4. The method of claim 3, wherein the compound is represented by formula Ia

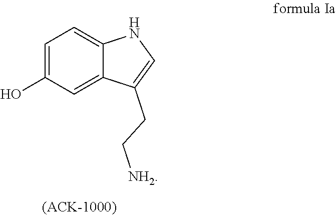 [SMILES: NCCc1c[nH]c2ccc(O)cc12]

5. The method of claim 1, wherein
R₁ is hydrogen, an alkyl group, an alcohol group, an alkoxy group or an alkyl ether group containing one or more oxyethylene units;
R₂ is hydrogen or hydroxyl; and
R₃ and R₄ together form a benzene ring fused to the nitrogen-containing bicyclic heteroring, wherein the benzene ring is substituted by —NH₂.

6. The method of claim 5, wherein
R₁ is an alkyl group, an alcohol group or an alkyl ether group containing one or more oxyethylene units;
R₂ is hydrogen; and
R₃ and R₄ together form a benzene ring fused to the nitrogen-containing bicyclic heteroring, wherein the benzene ring is substituted by —NH₂.

7. The method of claim 6, wherein the compound is represented by formula Ib 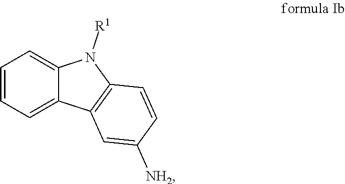
wherein R₁ is —CH₃, —CH₂CH₃, —CH₂CH₂CH₂CH₃, —CH₂CH₂OH, —CH₂CH₂CH₂OH or —CH₂CH₂(OCH₂CH₂)₄OCH₃.

8. The method of claim 7, wherein the compound is selected from the group consisting of

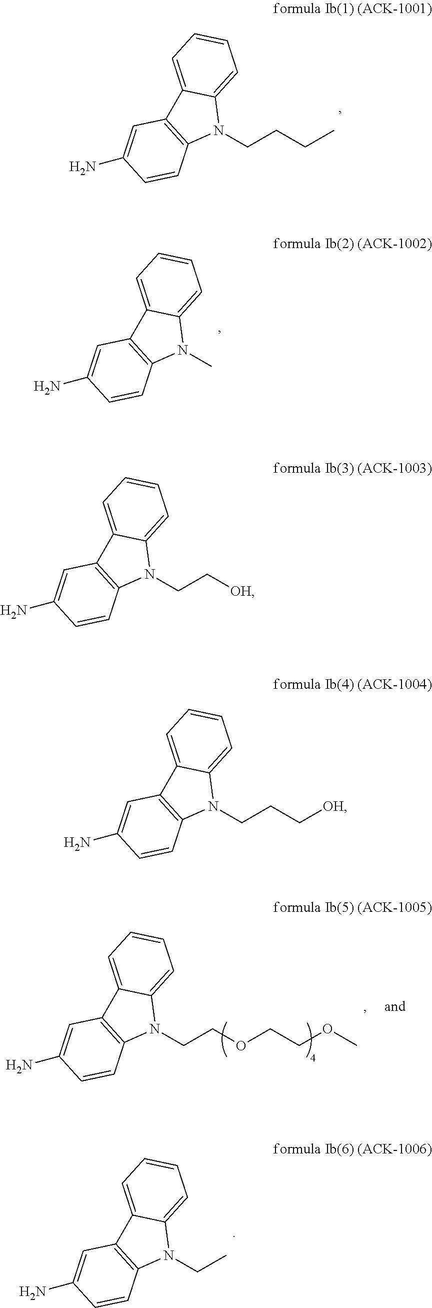

9. The method of claim 1, wherein the spectrometry technique comprises a tandem mass spectrometry (MS/MS) technique.

10. The method of claim 9, wherein the spectrometry technique comprises an LC-MS/MS technique.

11. The method of claim 1, wherein the biological sample is a liquid biological sample.

12. The method of claim 11, wherein the liquid biological sample is a serum sample or a urine sample.

13. The method of claim 1, wherein biological sample has a volume of 0.01 μL to 10 μL.

14. The method of claim 13, wherein the biological sample has a volume of 0.05 μL to 5 μL.

15. The method of claim 13, wherein the biological sample has a volume of 0.05 μL to 2 μL.

16. A method for determining ceruloplasmin activity and predicting the risk of a disease related to ceruloplasmin activity in a subject, comprising
(i) providing a biological sample which is obtained from the subject;
(ii) contacting the biological sample with a compound of formula I under a condition such that the compound is oxidized to produce an oxidized product of the compound, 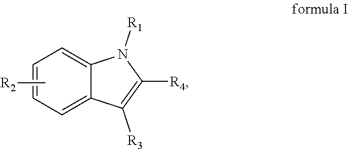
wherein
R₁ is hydrogen, an alkyl group, an alcohol group, an alkoxy group or an alkyl ether group containing one or more oxyethylene units:
R₂ is hydrogen or hydroxyl; and
R₃ and R₄, the same or different, are independently hydrogen or an alkylamine group, or
R₃ and R₄ together form a benzene ring fused to the nitrogen-containing bicyclic heteroring, wherein the benzene ring is substituted by —NH₂;
(iii) measuring a level of the oxidized product by a mass spectrometry technique; and
(iv) determining a level of ceruloplasmin activity and the likelihood of having a disease or condition related to ceruloplasmin activity of said subject based on the level of the oxidized product, wherein
a decreased level of the oxidized product below a reference level is indicative of deficiency of ceruloplasmin activity and an increased likelihood of having a disease or condition related to deficiency of ceruloplasmin activity; and/or
an increased level of the oxidized product above a reference level is indicative of excess of ceruloplasmin activity and an increased likelihood of having a disease or condition related to excess of ceruloplasmin activity.

17. The method of claim 16, wherein the disease or condition related to deficiency of ceruloplasmin activity is selected from the group consisting of aceruloplasminemia, Wilson's disease, Menke's disease, Alzheimer's disease and Parkinson's disease (PD).

18. The method of claim 16, wherein the disease or condition related to excess of ceruloplasmin activity includes Type II diabetes mellitus, valvular heart disease, coronary heart disease, bladder cancer, chronic obstructive pulmonary disease and kidney diseases.

19. The method of claim 16, wherein the subject is a fetus, a neonate, a child, an adolescent or an adult.

20. The method of claim 16, wherein the biological sample is a liquid biological sample.

21. The method of claim 20, wherein the liquid biological sample is a serum sample or a urine sample.

22. A kit, which comprises
a compound of formula I, 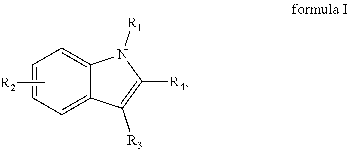
wherein
R₁ is hydrogen, an alkyl group, an alcohol group, an alkoxy group or an alkyl ether group containing one or more oxyethylene units;
R₂ is hydrogen or hydroxyl; and
R₃ and R₄, the same or different, are independently hydrogen or an alkylamine group, or
R₃ and R₄ together form a benzene ring fused to the nitrogen-containing bicyclic heteroring, wherein the benzene ring is substituted by —NH₂,
a reaction buffer,
a quenching agent and
instructions for using the kit for performing the method of determining ceruloplasmin activity as defined in claim 1.

23. (canceled)

24. (canceled)

25. (canceled)

26. A kit, which comprises
a compound of formula I, 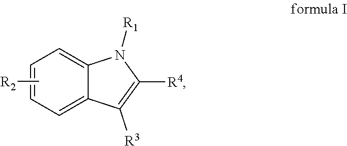
wherein
R₁ is hydrogen, an alkyl group, an alcohol group, an alkoxy group or an alkyl ether group containing one or more oxyethylene units;
R₂ is hydrogen or hydroxyl; and
R₃ and R₄, the same or different, are independently hydrogen or an alkylamine group, or
R₃ and R₄ together form a benzene ring fused to the nitrogen-containing bicyclic heteroring, wherein the benzene ring is substituted by —NH₂,
a reaction buffer,
a quenching agent and
instructions for using the kit for performing the method of predicting the risk of a disease related to ceruloplasmin activity in a subject as defined in claim 16.
